(19) United States
(12) Patent Application Publication
(10) Pub. No.: US 2010/0137396 A1
(43) Pub. Date: Jun. 3, 2010

(54) TREATMENT OF ESOPHAGEAL HIGH GRADE DYSPLASIA USING PHOTODYNAMIC THERAPY

(75) Inventors: Ravindra K. Pandey, Williamsville, NY (US); Thomas J. Dougherty, Grand Island, NY (US); David A. Bellnier, Buffalo, NY (US); Hector R. Nava, Buffalo, NY (US)

Correspondence Address: MICHAEL L. DUNN, SIMPSON & SIMPSON, PLLC, 5555 MAIN STREET, WILLIAMSVILLE, NY, 14221, US

(73) Assignee: Health Research, Inc, Buffalo, NY (US)

(21) Appl. No.: 12/448,660

(22) PCT Filed: Sep. 27, 2007
(86) PCT No.: PCT/US07/20816

§ 371 Date: Feb. 1, 2010

Related U.S. Application Data
(60) Provisional application No. 60/879,435, filed on Jan. 9, 2007.

(51) Int. Cl. A61K 31/407; A61P 35/00

(52) U.S. Cl. 514/410

(57) ABSTRACT

A method for treatment of esophageal high grade dysplasia comprising the steps of: injecting HPPH in a physiologically compatible medium into a patient having high grade dysplasia tissue to provide a dose level of 3 through 5 mg/m² of body surface area, waiting for a time period of 24 through 60 hours to permit preferential absorption of the HPPH into esophageal cancer tissue, and exposing the esophageal cancer tissue to light at a wavelength of about 670±5 nm at an energy of from about 75 to about 200 Joules/cm.

### CROSS REFERENCE TO RELATED PATENT APPLICATION

This application claims priority from U.S. Provisional Application No. 60/879,435, filed 09 Jan. 2007.

### STATEMENT REGARDING FEDERALLY SPONSORED RESEARCH

This invention was made with funding from the National Institute of Health Grant Numbers NIH (1R21 CA109914-01 and CA 55792). The United States Government may have certain rights in this invention.

### BACKGROUND OF THE INVENTION

High grade dysplasia, often associated with Barrett's esophagus, is considered a marker of greatly increased risk of developing esophageal cancer (Overholt et al., Gastrointestinal Endoscopy, volume 62, 488-498, 2005). Patients with Barrett's esophagus and associated high grade dysplasia have a >50% increased risk of developing esophageal cancer, necessitating aggressive treatment such as high risk surgery. Esophageal cancer is a debilitating and often deadly cancer causing irritation, pain, difficulty swallowing and partial and sometimes complete obstruction of the esophagus. “High grade dysplasia” is a disease characterized by significant changes in cellular morphology that often occur prior to development of invasive cancer.

For the last several years porphyrin-based compounds have been used for the treatment of certain cancers by photodynamic therapy (PDT). The concentration of certain porphyrins and related tetrapyrrolic systems is higher in malignant tumors than in most normal tissues and that has been one of the main reasons for using these molecules as photosensitizers. Some tetrapyrrole-based compounds have been effective in a wide variety of malignancies, including skin, lung, bladder, head and neck and esophagus. There have, however been associated problems with their use including prolonged skin phototoxicity, normal tissue damage, especially a high percentage of esophageal strictures, and insufficient depth of penetration. The precise mechanism(s) of PDT are unknown; however, the in vivo animal data suggest that both direct cell killing and loss of tumor vascular function play a significant role.

A well tested tetrapyrrolic compound for treatment of cancer is the 3-(1-hexyloxy) ethyl-derivative of pyropheophorbide-a (HPPH). HPPH, as used herein means the 3-(1-hexyloxy) ethyl-derivative of pyropheophorbide-a in both its free acid and ester and salt forms. This compound is tumor-avid and has undergone Phase I/II human clinical trials at the Roswell Park Cancer Institute in Buffalo, N.Y.

Photodynamic therapy (PDT) is believed to exploit the biological consequences of localized oxidative damage inflicted by photodynamic processes. Three critical elements are required for initial photodynamic processes to occur: a photosensitizer, light at photosensitizer-specific wavelength and oxygen. The light at the required wavelength is believed to trigger singlet oxygen production to destroy tissue in which it is concentrated.

Tetrapyrrolic compounds, such as porfimer sodium, sold under the trademark PHOTOFRIN™ has been found to be effective in the photodynamic treatment of high grade dysplasia and esophageal cancer ((Overholt et al., Gastrointestinal Endoscopy, volume 62, 488-498, 2005.

Unfortunately, the use of PHOTOFRIN™ to treat high grade dysplasia has a number of serious side effects including long term skin sensitivity to light, especially sunlight, and injury to surrounding normal tissue, such as esophageal strictures.

A review of published and non-published sources, e.g. Bellnier et al., Cancer Chemotherapeutic Pharmacology (2005) 57: 40-45 not necessarily prior art to the present invention, indicate that the use of porfimer sodium at a treatment concentration of 2 mg/kg can cause serious phototoxicity. Nevertheless, PHOTOFRIN™ at an optimized dose level of 2 mg/kg body weight and activation at its preferential light absorption wavelength of 630 nm, and optimized light exposure results in a first treatment complete cure rate of about 32 percent and a two treatment complete cure rate of about 58 percent and a three treatment complete cure rate of about 77 percent. Unfortunately, the treatment also results in significant damage to surrounding normal tissue. Normal tissue damage for PHOTOFRIN™, as measured by percent of strictures at only a single treatment is about 12% and using other reported data concerning PHOTOFRIN™ treatment, is greater than 20 percent based upon toxicity as determined by the presence of erythema, edema and necrosis.

The use of HPPH for treatment of obstructive esophageal cancer has been described. (Optical Methods for tumor Treatment and Detection: Mechanisms and Techniques in Photodynamic Therapy IX, Thomas Dougherty, Editor, Proceedings of SPIE Vol. 3909 (2000)). This document describes effects upon esophageal cancer at large doses of HPPH for treatment, i.e. 6 mg/m² or more.

### BRIEF DESCRIPTION OF THE INVENTION

In accordance with the invention, we have discovered that HPPH is effective against high grade dysplasia at significantly lower doses upon exposure of such tissue to light at the preferential absorption wavelength of HPPH (670±5 nm) at dose of from 3 to 5 mg/m² (0.5 to 0.13 mg/kg) of body surface area, and is more effective against high grade dysplasia with fewer side effects than PHOTOFRIN™.

HPPH, i.e. 3-(1-hexyloxy) ethyl-derivative of pyropheophorbide-a, has the following formula:

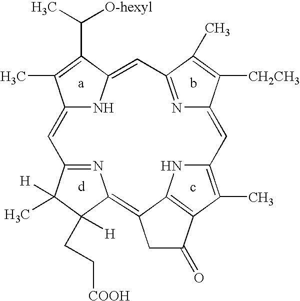

and includes the salts and alkyl esters thereof and may be prepared as set forth in U.S. Pat. Nos. 5,198,460 and 5,314,905 reissued as RE39094 and RE38994 respectively, all of which are incorporated herein by reference.

The method of the invention includes the steps of:
injecting HPPH in a physiologically compatible medium into a patient having high grade esophageal dysplasia to provide a dose level of 3 through 5 mg/m² of body surface area, preferably 3 through 4 mg/m² of body surface area.
waiting for a time period of 24 through 60 hours to permit preferential absorption of the HPPH into the high grade dysplasia tissue, and
exposing the esophageal high grade dysplasia tissue to light at a wavelength of about 670±5 nm at an energy of from about 75 to about 200 Joules/cm, preferably 75 to about 150 Joules/cm.

### DETAILED DESCRIPTION OF THE INVENTION

Injection of the HHPH is preferably accomplished intravenously usually over a time period of 0.75 to 3 hours in a physiologically compatible medium. The time period is functionally dependent upon rate of infusion and dose level desired The concentration is preferably 0.5-through 1.5 mg/ml in medium and the medium is preferably 0.1% polysorbate 80, 2% ethyl alcohol and 5% glucose in normal saline.

Exposure is accomplished using a fiber optic carrying laser light emitted by a laser. The laser may be any suitable laser emitting light at the wavelength and energy desired, e.g. a dye or diode laser. Exposure may be adjusted by length of time of exposure and/or adjustment of light intensity.

Using the above parameters, a phase I/II trial using HPPH and a phase III trial using PHOTOFRIN™, the latter approved by the United States Food and Drug Administration, the following results for response of high grade dysplasia were obtained, with CR being defined as complete ablation of high grade dysplasia.

Response After First Treatment

| PHOTOFRIN ™ | HPPH |
|---|---|
| 32% CR | 49% CR |

First Treatment Normal Tissue Damage

| | |
|---|---|
| 12% strictures | 3% strictures |

Response After Second Treatment

| | |
|---|---|
| 58% cure rate (observed) | not available |
| 54% expected cure rate (based upon | 74% expected cure rate |
| remaining uncured 68%) | (based upon 32% of |
| | 49% of remaining uncured 51%) |

Response After Third Treatment

| | |
|---|---|
| 77% cure rate (observed) | not available |
| 71% expected cure rate (based upon | 87% expected cure rate (based |
| 32% of remaining uncured 42%) | upon 49% of remaining |
| | uncured 26%) |

What is claimed is:

1. A method for treatment of high grade dysplasia comprising the steps of:
injecting HPPH in a physiologically compatible medium into a patient having high grade esophageal dysplasia tissue to provide a dose level of 3 through 5 mg/m² of body surface area,
waiting for a time period of 24 through 60 hours to permit preferential absorption of the HPPH into the high grade dysplasia tissue, and
exposing the esophageal high grade dysplasia tissue to light at a wavelength of about 670±5 nm at an energy of from about 75 to about 200 Joules/cm.

2. The method of claim 1 where the dose level of HPPH is 3-4 mg/m².

3. The method of claim 1 where the energy is from about 75 to about 150 Joules.

4. The method of claim 1 where the waiting time is from about 24 to about 60 hours.

5. The method of claim 1 where the HPPH is injected at a concentration of from about 0.5 through about 1.5 mg/ml in physiologically compatible medium.

6. The method of claim 5 where infusion time for the injection is from about 0.75 to about 3 hours.
